SEQUENCE LISTING (46 sequences)

SEQ ID NO: 1 - DNA, 63 bp, Artificial Sequence
  synthesized_CDR-L1 primer
  variation 31,32,34,35,37,38: /note=“n is A, T, C, or G”
accattacctgccgtgcgagccaggatgttnnknnknnkgtcgcatggtatcagcagaaacca

SEQ ID NO: 2 - DNA, 69 bp, Artificial Sequence
  synthesized_CDR-L2 primer
  variation 25,26,28,29,31,32,34,35,37,38: /note=“n is A, T, C, or G”
ggcaaagcgccgaaacttctgatannknnknnknnknnkctgtatagcggcgtgccgtcgcgtttttcg

SEQ ID NO: 3 - DNA, 69 bp, Artificial Sequence
  synthesized_CDR-L3 primer
  variation 28,29,31,32,34,35,37,38,43,44: /note=“n is A, T, C, or G”
gattttgcgacctactactgtcaacagnnknnknnknnkccgnnkaccttcggtcaaggcaccaaagtg

SEQ ID NO: 4 - DNA, 63 bp, Artificial Sequence
  synthesized_CDR-H1 primer
  variation 28,29,31,32,34,35,37,38: /note=“n is A, T, C, or G”
agctgtgcggcgagcgggttcaccattnnknnknnknnkattcattgggtgcgtcaagctccc

SEQ ID NO: 5 - DNA, 81 bp, Artificial Sequence
  synthesized_CDR-H2 primer
  variation 22,23,28,29,34,35,37,38,43,44,49,50: /note=“n is A, T, C, or G”
aaggggctggagtgggtcgcgnnkattnnkcccnnknnkggtnnkacannktatgccgacagcgtgaaaggtcgctttacg

SEQ ID NO: 6 - DNA, 66 bp, Artificial Sequence
  synthesized_CDR-H3 primer
  variation 25,26,28,29,31,32,34,35,37,38: /note=“n is A, T, C, or G”
acagcggtgtattattgcgcgcgtnnknnknnknnknnkgattattgggggcagggcacccttgtt

SEQ ID NO: 7 - protein, 11 aa, Artificial Sequence
  synthesized_CHS5_CDR-L1
RASQDVKEGVA

SEQ ID NO: 8 - protein, 8 aa, Artificial Sequence
  synthesized_CHS5_CDR-L2
DQSHRLYS

SEQ ID NO: 9 - protein, 9 aa, Artificial Sequence
  synthesized_CHS5_CDR-L3
QQYYAWPST

SEQ ID NO: 10 - protein, 13 aa, Artificial Sequence
  synthesized_CHS5_CDR-H1
AASGFTITDRTIH

SEQ ID NO: 11 - protein, 10 aa, Artificial Sequence
  synthesized_CHS5_CDR-H2
SIFPTKGVTT

SEQ ID NO: 12 - protein, 9 aa, Artificial Sequence
  synthesized_CHS5_CDR-H3
ARGRYWMDY

SEQ ID NO: 13 - protein, 11 aa, Artificial Sequence
  synthesized_CHS7_CDR-L1
RASQDVEDGVA

SEQ ID NO: 14 - protein, 8 aa, Artificial Sequence
  synthesized_CHS7_CDR-L2
GIKDLLYS

SEQ ID NO: 15 - protein, 9 aa, Artificial Sequence
  synthesized_CHS7_CDR-L3
QQYYRWPST

SEQ ID NO: 16 - protein, 13 aa, Artificial Sequence
  synthesized_CHS7_CDR-H1
AASGFTIDKEAIH

SEQ ID NO: 17 - protein, 10 aa, Artificial Sequence
  synthesized_CHS7_CDR-H2
SIYPHSGFTL

SEQ ID NO: 18 - protein, 8 aa, Artificial Sequence
  synthesized_CHS8_CDR-L2
DERERLYS

SEQ ID NO: 19 - protein, 9 aa, Artificial Sequence
  synthesized_CHS8_CDR-L3
QQYNTWPAT

SEQ ID NO: 20 - protein, 13 aa, Artificial Sequence
  synthesized_CHS8_CDR-H1
AASGFTISKASIH

SEQ ID NO: 21 - protein, 10 aa, Artificial Sequence
  synthesized_CHS8_CDR-H2
SIWPTKGFTT

SEQ ID NO: 22 - protein, 9 aa, Artificial Sequence
  synthesized_CHS8_CDR-H3
ARGRYWLDY

SEQ ID NO: 23 - protein, 11 aa, Artificial Sequence
  synthesized_ALA12_CDR-L1
RASQDVRDGVA

SEQ ID NO: 24 - protein, 8 aa, Artificial Sequence
  synthesized_ALA12_CDR-L2
DQMVRLYS

SEQ ID NO: 25 - protein, 9 aa, Artificial Sequence
  synthesized_ALA12_CDR-L3
QQYFNWPVT

SEQ ID NO: 26 - protein, 13 aa, Artificial Sequence
  synthesized_ALA12_CDR-H1
AASGFTIGNWTIH

SEQ ID NO: 27 - protein, 10 aa, Artificial Sequence
  synthesized_ALA12_CDR-H2
LIWPESGATL

SEQ ID NO: 28 - protein, 108 aa, Artificial Sequence
  synthesized_CHS5_VL domain
DIQMTQSPSSLSASVGDRVTITCRASQDVKEGVAWYQQKPGKAPKLLIDQSHRLYSGVPSRFSGSGSGTDFTLTISSLQPEDFATYYCQQYYAWPSTFGQGTKVEIKR

SEQ ID NO: 29 - protein, 116 aa, Artificial Sequence
  synthesized_CHS5_VH domain
EVQLVESGGGLVQPGGSLRLSCAASGFTITDRTIHWVRQAPGKGLEWVASIFPTKGVTTYADSVKGRFTISADTSKNTAYLQMNSLRAEDTAVYYCARGRYWMDYWGQGTLVTVSS

SEQ ID NO: 30 - protein, 108 aa, Artificial Sequence
  synthesized_CHS7_VL domain
DIQMTQSPSSLSASVGDRVTITCRASQDVEDGVAWYQQKPGKAPKLLIGIKDLLYSGVPSRFSGSGSGTDFTLTISSLQPEDFATYYCQQYYRWPSTFGQGTKVEIKR

SEQ ID NO: 31 - protein, 116 aa, Artificial Sequence
  synthesized_CHS7_VH domain
EVQLVESGGGLVQPGGSLRLSCAASGFTIDKEAIHWVRQAPGKGLEWVASIYPHSGFTLYADSVKGRFTISADTSKNTAYLQMNSLRAEDTAVYYCARGRYWMDYWGQGTLVTVSS

SEQ ID NO: 32 - protein, 108 aa, Artificial Sequence
  synthesized_CHS8_VL domain
DIQMTQSPSSLSASVGDRVTITCRASQDVKEGVAWYQQKPGKAPKLLIDERERLYSGVPSRFSGSGSGTDFTLTISSLQPEDFATYYCQQYNTWPATFGQGTKVEIKR

SEQ ID NO: 33 - protein, 116 aa, Artificial Sequence
  synthesized_CHS8_VH domain
EVQLVESGGGLVQPGGSLRLSCAASGFTISKASIHWVRQAPGKGLEWVASIWPTKGFTTYADSVKGRFTISADTSKNTAYLQMNSLRAEDTAVYYCARGRYWLDYWGQGTLVTVSS

SEQ ID NO: 34 - protein, 108 aa, Artificial Sequence
  synthesized_ALA12_VL domain
DIQMTQSPSSLSASVGDRVTITCRASQDVRDGVAWYQQKPGKAPKLLIDQMVRLYSGVPSRFSGSGSGTDFTLTISSLQPEDFATYYCQQYFNWPVTFGQGTKVEIKR

SEQ ID NO: 35 - protein, 116 aa, Artificial Sequence
  synthesized_ALA12_VH domain
EVQLVESGGGLVQPGGSLRLSCAASGFTIGNWTIHWVRQAPGKGLEWVALIWPESGATLYADSVKGRFTISADTSKNTAYLQMNSLRAEDTAVYYCARGRYWMDYWGQGTLVTVSS

SEQ ID NO: 36 - protein, 4 aa, Artificial Sequence
  synthesized_ER retention peptide
KDEL

SEQ ID NO: 37 - protein, 9 aa, Artificial Sequence
  synthesized_first polypeptide
ASAAGGSGT

SEQ ID NO: 38 - protein, 138 aa, Artificial Sequence
  synthesized_adaptor
YNKDQQSAFYEILNMPNLNEEQRNGFIQSLKDDPSQSTNVLGEAKKLNESQAPKADNGGGSGGGSGGGSGGGSGGSEVTIKVNLIFADGKIQTAEFKGTFEEATAEAYRYAALLAKVNGEYTADLEDGGNHMNIKFAG

SEQ ID NO: 39 - protein, 11 aa, Artificial Sequence
  synthesized_M9_CDR-L1
RASQDVNDGVA

SEQ ID NO: 40 - protein, 8 aa, Artificial Sequence
  synthesized_M9_CDR-L2
SGSPWLYS

SEQ ID NO: 41 - protein, 9 aa, Artificial Sequence
  synthesized_M9_CDR-L3
QQYFNWPIT

SEQ ID NO: 42 - protein, 13 aa, Artificial Sequence
  synthesized_M9_CDR-H1
AASGFTIDNYGIH

SEQ ID NO: 43 - protein, 10 aa, Artificial Sequence
  synthesized_M9_CDR-H2
WIWPYGGSTY

SEQ ID NO: 44 - protein, 9 aa, Artificial Sequence
  synthesized_M9_CDR-H3
ARGYYWYDY

SEQ ID NO: 45 - protein, 5 aa, Artificial Sequence
  synthesized_type I amino acid sequence
STNQG

SEQ ID NO: 46 - protein, 5 aa, Artificial Sequence
  synthesized_type II amino acid sequence
KRHDE

(19) United States
(12) Patent Application Publication
(10) Pub. No.: US 2024/0277865 A1
(43) Pub. Date: Aug. 22, 2024

(54) A PHAGE-DISPLAYED SINGLE-CHAIN VARIABLE FRAGMENT LIBRARY FOR SELECTING ANTIBODY FRAGMENTS SPECIFIC TO MESOTHELIN

(71) Applicant: Academia Sinica, Taipei City (TW)

(72) Inventors: An-Suei YANG, Taipei City (TW); Hung-Ju HSU, Taipei City (TW); Chao-Ping TUNG, Taipei City (TW); Chung-Ming YU, New Taipei City (TW); Chi-Yung CHEN, Taipei City (TW); Hong-Sen CHEN, Taipei City (TW); Yu-Chuan HUANG, Taipei City (TW); Pei-Hsun TSAI, Taipei City (TW); Szu-Yu LIN, Taipei City (TW); Hung-Pin PENG, Taipei City (TW)

(21) Appl. No.: 18/567,765

(22) PCT Filed: Jun. 8, 2022
(86) PCT No.: PCT/US2022/032746

§ 371 Date: Dec. 6, 2023

Related U.S. Application Data
(60) Provisional application No. 63/208,496, filed on Jun. 8, 2021.

(51) Int. Cl. A61K 47/68; A61K 39/00; A61P 35/00; C07K 16/00; C07K 16/30

(52) U.S. Cl. CPC A61K 47/6851; A61K 47/68031; A61K 47/6829; A61P 35/00; C07K 16/005; C07K 16/30; A61K 2039/505; C07K 2317/565; C07K 2317/622; C07K 2317/73

(57) ABSTRACT

Disclosed herein is a phage-displayed single-chain variable fragment (scFv) library, which comprises a plurality of phage-displayed scFvs characterized with a specific sequence in each CDR. The present phage-displayed scFv library is useful in selecting an antibody fragment exhibiting a binding affinity and specificity to mesothelin (MSLN). Also disclosed herein are a recombinant antibody specific to MSLN, an immunoconjugate comprising the recombinant antibody, and uses thereof in treating cancers.

### BACKGROUND OF THE INVENTION

### 1. Field of the Invention

The present disclosure in general relates to the field of antibody fragment library. More particularly, the present disclosure relates to a phage-displayed single-chain variable fragment (scFv) library and the uses thereof in selecting antibody fragments for treating cancers, especially mesothelin (MSLN)-positive cancers.

### 2. Description of Related Art

Targeting MSLN is an attractive cancer therapeutic strategy, which has led to a large number of clinical trials of treating diverse cancers. MSLN is known to be overexpressed in several malignant tumor cells. At molecular level, the interaction of MSLN with cancer antigen 125 (CA-125; also known as Mucin 16 (MUC16)), which participates in cell-to-cell interactions enabling tumorigenesis and tumor proliferation, increases the motility and invasion of pancreatic carcinoma cells. The overexpression of MSLN activates nuclear factor kappa-light-chain-enhancer of activated B cells (NF-κB), mitogen-activated protein kinase (MAPK), and phosphatidylinositol-3 kinase (PI3K) pathways, leading to resistance of apoptosis in pancreatic cancer cells. Also, MSLN overexpression results in MMP-7 activation associated with pancreatic carcinoma cell invasion, and correlates with higher MMP-9 expression in malignant pleural mesothelioma, promoting tumor invasion. Clinical data indicate that elevated MSLN expression is associated with increase in tumor burden and poor overall survival in patients of various cancers, while MSLN's normal expression is limited to mesothelial cells, which are dispensable without substantial adversary side effect. As such, MSLN may serve as a candidate of targeted therapy for different cancers.

Antibody-drug conjugates (ADCs) targeting MSLN have been demonstrated to be a viable strategy in treating MSLN-positive cancers. However, developing antibodies as targeting modules in ADCs for toxic payload delivery to the tumor site but not to normal tissues is not a straightforward task with many potential hurdles. In principle, successful ADC development should meet the following minimal criteria: (1) feasibility of preparing the ADCs with sufficient yield; (2) appropriate affinity and specificity of the ADCs binding to the target antigen; (3) ADC binding to the antigen on the epitope accessible for ADC binding in biologically relevant state; (4) ADC internalization in cells following binding to an appropriate epitope; and (5) release of toxic payload after the receptor-mediated endocytosis of the ADC-antigen complex. In practice, antibody candidates simultaneously satisfying all the criteria above are difficult to attain.

In view of the foregoing, there exists in the related art a need for a method of efficiently producing an antibody with sufficient specificity and/or affinity to MSLN so as to develop an ADC for treating cancers, especially MSLN-positive cancers.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The first aspect of the present disclosure is directed to a phage-displayed scFv library comprising a plurality of phage-displayed scFvs. In structure, each phage-displayed scFv comprises a first light chain complementarity determining region (CDR-L1), a second light chain CDR (CDR-L2), a third light chain CDR (CDR-L3), a first heavy chain CDR (CDR-H1), a second heavy chain CDR (CDR-H2) and a third heavy chain CDR (CDR-H3). According to embodiments of the present disclosure, the CDR-L1 is encoded by a first coding sequence comprising the nucleic acid sequence of SEQ ID NO: 1; the CDR-L2 is encoded by a second coding sequence comprising the nucleic acid sequence of SEQ ID NO: 2; the CDR-L3 is encoded by a third coding sequence comprising the nucleic acid sequence of SEQ ID NO: 3; the CDR-H1 is encoded by a fourth coding sequence comprising the nucleic acid sequence of SEQ ID NO: 4; the CDR-H2 is encoded by a fifth coding sequence comprising the nucleic acid sequence of SEQ ID NO: 5; and the CDR-H3 is encoded by a sixth coding sequence comprising the nucleic acid sequence of SEQ ID NO: 6.

According to certain embodiments, the phage of the present phage-displayed scFv library is M13 phage or T7 phage. In some working examples, the phage is M13 phage.

According to some embodiments, the phage-displayed scFv library comprises a first, a second, a third and a fourth phage-displayed scFvs, wherein the CDR-L1, CDR-L2, CDR-L3, CDR-H1, CDR-H2 and CDR-H3 of the first phage-displayed scFv respectively comprise the amino acid sequences of SEQ ID NOs: 7, 8, 9, 10, 11 and 12; the CDR-L1, CDR-L2, CDR-L3, CDR-H1, CDR-H2 and CDR-H3 of the second phage-displayed scFv respectively comprise the amino acid sequences of SEQ ID NOs: 13, 14, 15, 16, 17 and 12; the CDR-L1, CDR-L2, CDR-L3, CDR-H1, CDR-H2 and CDR-H3 of the third phage-displayed scFv respectively comprise the amino acid sequences of SEQ ID NOs: 7, 18, 19, 20, 21 and 22; and the CDR-L1, CDR-L2, CDR-L3, CDR-H1, CDR-H2 and CDR-H3 of the fourth phage-displayed scFv respectively comprise the amino acid sequences of SEQ ID NOs: 23, 24, 25, 26, 27 and 12.

According to some embodiments of the present disclosure, at least one of the plurality of phage-displayed scFvs of the present scFv library is specific to MSLN.

The second aspect of the present disclosure pertains to a recombinant antibody specific to MSLN. According to some embodiments, the recombinant antibody is derived from the present phage-displayed scFv library, and in structure comprises a light chain variable (VL) domain and a heavy chain variable (VH) domain, in which the VL domain comprises a CDR-L1, a CDR-L2 and a CDR-L3; and the VH domain comprises a CDR-H1, a CDR-H2 and a CDR-H3.

According to some embodiments, the CDR-L1, CDR-L2, CDR-L3, CDR-H1, CDR-H2 and CDR-H3 respectively comprise the amino acid sequences of SEQ ID NOs: 7-12. According to some preferred embodiments, the VL domain comprises an amino acid sequence at least 85% identical to SEQ ID NO: 28, and the VH domain comprises an amino acid sequence at least 85% identical to SEQ ID NO: 29. In some working examples, the VL and VH domains respectively comprise amino acid sequences 100% identical to SEQ ID NOs: 28 and 29.

According to certain embodiments, the CDR-L1, CDR-L2 and CDR-L3 respectively comprise the amino acid sequences of SEQ ID NOs: 13-15, and the CDR-H1, CDR-H2 and CDR-H3 respectively comprise the amino acid sequences of SEQ ID NOs: 16, 17 and 12. According to some preferred embodiments, the VL domain comprises an amino acid sequence at least 85% identical to SEQ ID NO: 30, and the VH domain comprises an amino acid sequence at least 85% identical to SEQ ID NO: 31. In some working examples, the VL and VH domains respectively comprise amino acid sequences 100% identical to SEQ ID NOs: 30 and 31.

According to alternative embodiments, the CDR-L1, CDR-L2 and CDR-L3 respectively comprise the amino acid sequences of SEQ ID NOs: 7, 18 and 19, and the CDR-H1, CDR-H2 and CDR-H3 respectively comprise the amino acid sequences of SEQ ID NOs: 20-22. According to some preferred embodiments, the VL domain comprises an amino acid sequence at least 85% identical to SEQ ID NO: 32, and the VH domain comprises an amino acid sequence at least 85% identical to SEQ ID NO: 33. In some working examples, the VL and VH domains respectively comprise amino acid sequences 100% identical to SEQ ID NOs: 32 and 33.

According to some embodiments, the CDR-L1, CDR-L2 and CDR-L3 respectively comprise the amino acid sequences of SEQ ID NOs: 23-25, and the CDR-H1, CDR-H2 and CDR-H3 respectively comprise the amino acid sequences of SEQ ID NOs: 26, 27 and 12. According to some preferred embodiments, the VL domain comprises an amino acid sequence at least 85% identical to SEQ ID NO: 34, and the VH domain comprises an amino acid sequence at least 85% identical to SEQ ID NO: 35. In some working examples, the VL and VH domains respectively comprise amino acid sequences 100% identical to SEQ ID NOs: 34 and 35.

Depending on desired purpose, the recombinant antibody may be present in the form of an intact antibody (e.g., an immunoglobulin) or an antibody fragment comprising the VL and VH domains, such as an scFv or a fragment antigen-binding (Fab).

The recombinant antibody is useful in preparing an ADC for treating cancers (e.g., MSLN-positive cancers). Accordingly, the third aspect of the present disclosure pertains to an immunoconjugate that targets MSLN. In structure, the immunoconjugate comprises a present recombinant antibody, a functional motif, and a linker connecting the recombinant antibody to the functional motif.

According to some embodiments, the functional motif comprises an immunotoxin, such as an exotoxin (e.g., Pseudomonas Exotoxin (PE) A or the derivative thereof). In certain exemplary embodiments, the exotoxin is a truncated form of PE A subunit toxin. Optionally, the functional motif further comprises an endoplasmic reticulum (ER) retention peptide connected with the immunotoxin. According to some working examples, the ER retention peptide comprises the amino acid sequence of “KDEL” (SEQ ID NO: 36) and is disposed at the C-terminus of the PE A subunit toxin.

According to alternative embodiments, functional motif comprises a cytotoxic drug, for example, auristatin or a derivative thereof. In one working example, the cytotoxic drug is monomethyl auristatin E (MMAE).

The linker of the present immunoconjugate may be, (1) a valine-citrulline dipeptide; (2) a first polypeptide comprising the amino acid sequence of SEQ ID NO: 37; or (3) an adaptor comprising at least one AL module, wherein each AL module comprises a protein A fragment at the N-terminus, a protein L fragment at the C-terminus, and a second polypeptide connecting the protein A and protein L fragments. According to one specific example, the adaptor comprises the amino acid sequence of SEQ ID NO: 38.

Another aspect of the present disclosure pertains to a pharmaceutical composition that comprises an immunoconjugate according to any embodiment of the present disclosure, and a pharmaceutically acceptable excipient.

Also disclosed herein is a method of treating a cancer in a subject. The method comprises administering to the subject an effective amount of an immunoconjugate or a pharmaceutical composition in accordance with any aspect and embodiment of the present disclosure. According to some embodiments, the cancer has MSLN expressed thereon, i.e., a being an MSLN-positive cancer.

The cancer treatable with the present method may be any of gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma. According to one embodiment, the cancer is a gastric cancer. According to another embodiment, the cancer is a pancreatic cancer.

The subject is a mammal, preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A and 1B respectively depict the treatments of N87 and Capan-2 xenograft mouse models with anti-MSLN IgG1-vcMMAEs according to one example of the present disclosure. Four IgG1-vcMMAEs (including CHS5-vcMMAE, CHS7-vcMMAE, CHS8-vcMMAE, and ALA12-vcMMAE) of the experimental ADC group were used for the in vivo treatments of the N87 (FIG. 1A) and Capan-2 (FIG. 1B) xenograft mouse models, and the treatment results were compared with those of the positive ADC control (SS1-vcMMAE), isotype ADC control (S40-vcMMAE) and vehicle control. Two-tailed paired Student t-test P-values indicated statistical significance (*p<0.05).

FIGS. 2A and 2B respectively depict bio-distributions of the anti-MSLN IgG1s conjugated with a fluorescent dye (DYLIGHT™ 680) in N87 (FIG. 2A) and Capan-2 (FIG. 2B) xenograft models according to one example of the present disclosure. The mean values and standard deviations of bio-distributions were calculated with three mice in each of the experimental groups.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “antibody” is used in the broadest sense and specifically covers monoclonal antibodies (including full length monoclonal antibodies), polyclonal antibodies, multi-specific or multivalent antibodies (e.g., bi-specific antibodies), and antibody fragments so long as they exhibit the desired biological activity. The term “antibody fragment” or “the fragment of an antibody” refers to a portion of a full-length antibody, generally the antigen binding or variable domain (i.e., VL and VH domains) of a full-length antibody. Examples of the antibody fragment include fragment antigen-binding (Fab), Fab′, F(ab′)₂, single-chain variable fragment (scFv), diabody, linear antibody, single-chain antibody molecule, and multi-specific antibody formed from antibody fragments.

The term “antibody library” refers to a collection of antibodies and/or antibody fragments displayed for screening and/or combination into full antibodies. The antibodies and/or antibody fragments may be displayed on a ribosome; on a phage; or on a cell surface, in particular a yeast cell surface.

As used herein, the term “single-chain variable fragment” or “scFv” is a fusion protein comprising the variable domains of the heavy (VH) and light chains (VL) of an immunoglobulin, in which the VH and VL are covalently linked to form a VH::VL heterodimer. The VH and VL are either joined directly or joined by a peptide-encoding linker, which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences.

The term “complementarity determining region” (CDR) used herein refers to the hypervariable region of an antibody molecule that forms a surface complementary to the 3-dimensional surface of a bound antigen. Proceeding from N-terminus to C-terminus, each of the antibody heavy and light chains comprises three CDRs (i.e., CDR-L1, CDR-L2 and CDR-L3 comprised in the light chain, and CDR-H1, CDR-H2 and CDR-H3 comprised in the heavy chain). A HLA-DR antigen-binding site, therefore, includes a total of six CDRs that comprise three CDRs from the variable region of a heavy chain and three CDRs from the variable region of a light chain. The amino acid residues of CDRs are in close contact with bound antigen, wherein the closest antigen contact is usually associated with the heavy chain CDR3.

As used herein, the term “variable domain” or “variable region” of an antibody refers to the amino-terminal regions of heavy or light chain of the antibody. These regions are generally the most variable parts of an antibody and contain the antigen-binding sites. The term “variable” refers to the fact that certain portions of the variable domains differ extensively in sequence among antibodies, and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprises four FR regions, largely adopting a beta-sheet configuration, connected by three CDRs, which form loops connecting, and in some cases forming part of, the beta-sheet structure. The CDRs in each chain are held together in close proximity by the FR regions, and with the CDRs from the other chain, contribute to the formation of the antigen-binding site of antibodies. The constant regions are not involved directly in binding an antibody to an antigen, but exhibit various effector functions, such as participation of the antibody in antibody-dependent cellular toxicity.

“Percentage (%) sequence identity” with respect to any amino acid sequence identified herein is defined as the percentage of amino acid residues in a candidate sequence that are identical with the amino acid residues in the specific reference sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage sequence identity of a given sequence A to a subject sequence B (which can alternatively be phrased as a given sequence A that has a certain % sequence identity to a given sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in the subject sequence B.

As discussed herein, minor variations in the amino acid sequences of antibodies are contemplated as being encompassed by the presently disclosed and claimed inventive concept(s), providing that the variations in the amino acid sequence maintain at least 85% sequence identity, such as at least 85%, 86%, 87%, 88%, 89%, 90%, 91%, 92%, 93%, 94%, 95%, 96%, 97%, 98% and 99% sequence identity. Antibodies of the present disclosure may be modified specifically to alter a feature of the peptide unrelated to its physiological activity. For example, certain amino acids can be changed and/or deleted without affecting the physiological activity of the antibody in this study (i.e., the ability of binding to coronavirus). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of antibodies can be readily prepared by those of ordinary skill in the art. Preferred amino- and carboxyl-termini of fragments or analogs occur near boundaries of functional regions.

The term “phagemid” refers to a vector, which combines attributes of a bacteriophage and a plasmid. A bacteriophage is defined as any one of a number of viruses that infect bacteria.

The terms “nucleic acid sequence”, “nucleotide sequence” or “nucleic acid” can be used interchangeably and are understood to mean, according to the present disclosure, either a double-stranded DNA, a single-stranded DNA or a product of transcription of said DNA (e.g., RNA molecule). It should also be understood that the present disclosure does not relate to genomic polynucleic acid sequences in their natural environment or natural state. The nucleic acid, polynucleotide, or nucleic acid sequences of the invention can be isolated, purified (or partially purified), by separation methods including, but not limited to, ion-exchange chromatography, molecular size exclusion chromatography, or by genetic engineering methods such as amplification, subtractive hybridization, cloning, sub-cloning or chemical synthesis, or combinations of these genetic engineering methods.

The terms “coding sequence” as used herein refers to nucleotide sequences and nucleic acid sequences, including both RNA and DNA, that encode genetic information for the synthesis of a RNA, a protein, or any portion of a RNA or protein. Nucleotide sequences that are not naturally part of a particular organism's genome are referred to as “foreign nucleotide sequences”, “heterologous nucleotide sequences”, or “exogenous nucleotide sequences”. “Heterologous proteins” are proteins encoded by foreign, heterologous or exogenous nucleotide sequences and therefore are often not naturally expressed in the cell. A nucleotide sequence that has been isolated and then reintroduced into the same type (e.g., same species) of organism is not considered to be a naturally occurring part of a particular organism's genome and is therefore considered exogenous or heterologous.

The term “subject” refers to a mammal including the human species suitable to be treated by the antibody, immunoconjugate, pharmaceutical composition and/or method of the present disclosure. The term “subject” is intended to refer to both the male and female gender unless one gender is specifically indicated.

### II. Description of the Invention

The object of the present disclosure aims at providing a phage-displayed scFv library for the development of an antibody, which exhibits binding affinity and/or specificity to a tumor-associated antigen (TAA), e.g., MSLN, and accordingly may be conjugated with a therapeutic agent to target and treat cancers, e.g., the MSLN-positive cancers.

### (II-1) Phage-Displayed Sc Fv Library

The first aspect of the present disclosure is thus directed to a phage-displayed scFv library, which comprises a plurality of phage-displayed scFvs. In structure, each scFv comprises three CDRs (i.e., CDR-H1, CDR-H2, and CDR-H3) in the heavy chain thereof, and three CDRs (i.e., CDR-L1, CDR-L2, and CDR-L3) in the light chain thereof. According to some embodiments of the present disclosure, the CDR-L1 is encoded by a first coding sequence comprising the nucleic acid sequence of SEQ ID NO: 1, the CDR-L2 is encoded by a second coding sequence comprising the nucleic acid sequence of SEQ ID NO: 2, the CDR-L3 is encoded by a third coding sequence comprising the nucleic acid sequence of SEQ ID NO: 3, the CDR-H1 is encoded by a fourth coding sequence comprising the nucleic acid sequence of SEQ ID NO: 4, the CDR-H2 is encoded by a fifth coding sequence comprising the nucleic acid sequence of SEQ ID NO: 5, and the CDR-H3 is encoded by a sixth coding sequence comprising the nucleic acid sequence of SEQ ID NO: 6.

According to the IUPAC code, in the nucleotide sequences of SEQ ID NOs: 1-6, A represents adenine; C represents cytosine; G represents guanine; T represents thymine; B represents any nucleotide of C, G or T; D represents any nucleotide of A, T, or G; H represents any nucleotide of A, C, or T; K represents nucleotide G or T; M represents A or C; N represents any nucleotide of A, T, C, or G; R represents nucleotide A or G; S represents nucleotide G or C; V represents any nucleotide of A, C, or G; W represents nucleotide A or T; and Y represents nucleotide C or T.

According to some embodiments, the phage-displayed scFv library comprises at least four phage-displayed scFvs (i.e., a first, a second, a third and a fourth phage-displayed scFvs), each of the phage-displayed scFvs comprises CDRs respectively encoded by the first to sixth coding sequences. According to the embodiments, the CDR-L1, CDR-L2, CDR-L3, CDR-H1, CDR-H2 and CDR-H3 of the first phage-displayed scFv respectively comprise the amino acid sequences of SEQ ID NOs: 7, 8, 9, 10, 11 and 12; the CDR-L1, CDR-L2, CDR-L3, CDR-H1, CDR-H2 and CDR-H3 of the second phage-displayed scFv respectively comprise the amino acid sequences of SEQ ID NOs: 13, 14, 15, 16, 17 and 12; the CDR-L1, CDR-L2, CDR-L3, CDR-H1, CDR-H2 and CDR-H3 of the third phage-displayed scFv respectively comprise the amino acid sequences of SEQ ID NOs: 7, 18, 19, 20, 21 and 22; and the CDR-L1, CDR-L2, CDR-L3, CDR-H1, CDR-H2 and CDR-H3 of the fourth phage-displayed scFv respectively comprise the amino acid sequences of SEQ ID NOs: 23, 24, 25, 26, 27 and 12.

The phage-displayed scFv library is constructed with protein A and protein L selection, so that each of the plurality of phage-displayed scFvs has a VH domain capable of binding to protein A, and a VL domain capable of binding to protein L. Further, the phage-displayed scFv library is also subjected to a target antigen (e.g., TAA, such as MSLN) selection so as to ensure that each of the plurality of phage-displayed scFvs exhibits a binding affinity and/or specificity to the target antigen (e.g., TAA, such as MSLN). The phage-displayed scFv library may be constructed in accordance with the method described in U.S. Pat. No. 10,336,816 B2.

According to some embodiments, the phage for displaying the scFv is M13 phage or T7 phage. Preferably, each phage of the present scFv library harbors one single phagemid.

According to one embodiment, the scFvs displayed by the present scFv library are well-folded; particularly, they can be expressed on phage surfaces, or secreted as soluble form.

(II-2) Selecting Antibody Fragments from the Phage-Displayed Sc Fv Library

The established scFv library provides a means to effectively identify an antibody fragment exhibiting a binding affinity and/or specificity to the target antigen (e.g., TAA, such as MSLN). The method of selecting an antibody fragment specific to the target antigen from the scFv library comprises the steps of,
(a) exposing the phage-displayed scFv library to the target antigen;
(b) selecting, from the phage-displayed scFv library of the step (a), a plurality of phages that respectively express scFvs exhibiting binding affinity to the target antigen;
(c) respectively enabling the plurality of phages selected in the step (b) to express a plurality of soluble scFvs;
(d) exposing the plurality of soluble scFvs of the step (c) to the target antigen;
(e) determining the respective binding affinity of the plurality of soluble scFvs to the target antigen in the step (d); and
(f) based on the results determined in the step (e), selecting one soluble scFv that exhibits superior affinity over the other soluble scFvs of the plurality of soluble scFvs to the target antigen as the antibody fragment.

In the step (a), the scFv library is exposed to the target antigen or the fragment thereof. According to some embodiments, the target antigen is MSLN.

In the step (b), a plurality of phages respectively expressing scFvs that exhibit binding affinity to the target antigen (e.g., MSLN) are selected from the scFv library. Specifically, the product of the step (b) is subjected to an elution buffer, which generally is an acidic solution (such as glycine solution, pH 2.2), so as to disrupt the binding between the target antigen and phage-display scFv. By this way, the plurality of phages that respectively express scFvs exhibiting binding affinity to the target antigen are collected.

Optionally, the step (b) is carried out under an acidic condition. Specifically, the product of the step (b) may be subjected to an acidic treatment (for example, a washing buffer having a pH value ranging between 5-7, such as pH 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7; preferably, a washing buffer having a pH value of 5.0) followed by the afore-mentioned elution step to collect the plurality of phages.

Next, in the step (c), the plurality of phages selected in the step (b) are subjected to conditions that enable them to produce a plurality of soluble scFvs. This step can be carried out by using methods known to any person having ordinary skill in the art. According to certain embodiments of the present disclosure, the expression of VH and VL domains may be driven by a lactose operon (lac operon); as known by one skilled artisan, the lac operon would be induced by isopropyl-thio-B-D-galactoside (IPTG), which then drives the expression of the down-stream genes (i.e., genes encoding the VH and VL domains). The produced scFv are then secreted into the supernatant of culture medium and could be collected therefrom.

In the step (d), the soluble scFvs produced in the step (c) are respectively mixed with the target antigen so as to form antigen-scFv complexes.

Then, in the step (e), the level of the antigen-scFv complexes formed in the step (d) is determined by a method known to a person having ordinary skill in the art for analyzing the binding affinity of two molecules (e.g., the binding affinity of an antibody to an antigen); for example, ELISA, western blotting (WB) assay, flow cytometry, surface plasmon resonance (SPR), or LFIA. In general, the level of the antigen-scFv complexes is proportional to the binding affinity of the scFv to the target antigen.

Finally, in the step (f), the antibody fragment is selected based on the binding affinity determined in the step (e). More specifically, the soluble scFv that exhibits superior affinity to the target antigen (e.g., MSLN) over the other soluble scFvs of the plurality of soluble scFvs is selected as the antibody fragment.

According to optional embodiments, the method further comprises the step of exposing the plurality of soluble scFvs of step (c) to protein A and/or protein L prior to the step (d) thereby selecting soluble scFvs exhibiting binding affinity to protein A and/or protein L. According to alternative embodiments, the method further comprises the step of exposing the soluble scFv of step (f) to protein A and/or protein L after the step (f) so as to ensure that the selected soluble scFv exhibit a binding affinity to protein A and/or protein L.

According to some embodiments of the present disclosure, four antibody fragments, respectively designated as “CHS5 scFv”, “CHS7 scFv”, “CHS8 scFv” and “ALA12 scFv”, are selected from different rounds of selection.

### (II-3) Recombinant Antibodies or the Fragment Thereof

The antibody fragment selected from the present scFv library can be employed to produce an intact antibody (e.g., an IgG antibody) or different types of antibody fragment (e.g., a Fab, Fab′ or F(ab′)₂). The method for producing the intact antibody or antibody fragment are known in the art, for example, the method described in U.S. Pat. No. 10,336,815 B2 or 10,336,816 B2. According to some embodiments of the present disclosure, four intact antibodies are produced in the form of recombinant IgG antibody, which, in addition to the VL and VH domains of the selected scFv, further comprises a light chain constant (CL) domain and a heavy chain constant (CH) domain of an immunoglobulin; in these embodiments, the recombinant IgG antibodies are respectively designated as “CHS5 IgG”, “CHS7 IgG”, “CHS8 IgG” and “ALA12 IgG” . . . . The thus-produced recombinant antibody and its fragment (e.g., scFv, Fab, Fab′ or F(ab′)2) are included in the scope of the present disclosure.

As could be appreciated, the present antibody (including the recombinant IgG antibody and its fragment) may alternatively be produced by DNA cloning. Specifically, based on the present disclosure, a skilled artisan may construct a DNA expression vector comprising the CDR sequences of the present antibody, followed by introducing the constructed DNA expression vector into a host cell, such as E. Coli cell, simian COS cell, Chinese hamster ovary (CHO) cell or myeloma cell that do not produce immunoglobulin proteins, so as to synthesize the desired antibody in the host cell.

According to embodiments of the present disclosure, each antibody comprises a VL and a VH domains, each of which comprises three CDRs, i.e., CDR-L1, CDR-L2 and CDR-L3 in the VL domain, and CDR-H1, CDR-H2 and CDR-H3 in the VH domain. The CDR sequences of the present antibodies or their fragments are summarized in Table 1.

TABLE 1
Amino acid sequences of specified antibody

| | CHS5 | CHS7 | CHS8 | ALA12 |
|---|---|---|---|---|
| | (SEQ ID NO) | (SEQ ID NO) | (SEQ ID NO) | (SEQ ID NO) |
| CDR-L1 | RASQDVKEGVA | RASQDVEDGVA | RASQDVKEGVA | RASQDVRDGVA |
| | (7) | (13) | (7) | (23) |
| CDR-L2 | DQSHRLYS | GIKDLLYS | DERERLYS | DQMVRLYS |
| | (8) | (14) | (18) | (24) |
| CDR-L3 | QQYYAWPST | QQYYRWPST | QQYNTWPAT | QQYFNWPVT |
| | (9) | (15) | (19) | (25) |
| CDR-H1 | AASGFTITDRTIH | AASGFTIDKEAIH | AASGFTISKASIH | AASGFTIGNWTIH |
| | (10) | (16) | (20) | (26) |
| CDR-H2 | SIFPTKGVTT | SIYPHSGFTL | SIWPTKGFTT | LIWPESGATL |
| | (11) | (17) | (21) | (27) |
| CDR-H3 | ARGRYWMDY | ARGRYWMDY | ARGRYWLDY | ARGRYWMDY |
| | (12) | (12) | (22) | (12) |

According to certain embodiments, the VL domain of the present CHS5 antibody (e.g., CHS5 IgG or CHS5 scFv) comprises an amino acid sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 28, and the VH domain of the present CHS5 antibody (e.g., CHS5 IgG or CHS5 scFv) comprises an amino acid sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 29. As would be appreciated, the sequence (e.g., the framework sequence) of the VL and VH domains may vary (e.g., being substituted by conserved or non-conserved amino acid residues) without affecting the binding affinity and/or specificity of the present antibody. Preferably, the sequence(s) of the VL and VH domains is/are conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue). According to some preferred embodiments, the VL and VH domains of the present CHS5 antibody (e.g., CHS5 IgG or CHS5 scFv) respectively comprise amino acid sequences at least 90% identical to SEQ ID NOs: 28 and 29. More preferably, the VL and VH domains of the present CHS5 antibody (e.g., CHS5 IgG or CHS5 scFv) respectively comprise amino acid sequences at least 95% identical to SEQ ID NOs: 28 and 29. In one exemplary embodiment, the VL domain of the present CHS5 antibody (e.g., CHS5 IgG or CHS5 scFv) comprises the amino acid sequence of SEQ ID NO: 28 (i.e., comprising an amino acid sequence 100% identical to SEQ ID NO: 28), and the VH domain of the present CHS5 antibody (e.g., CHS5 IgG or CHS5 scFv) comprises the amino acid sequence of SEQ ID NO: 29 (i.e., comprising an amino acid sequence 100% identical to SEQ ID NO: 29).

According to some embodiments, the VL domain of the present CHS7 antibody (e.g., CHS7 IgG or CHS7 scFv) comprises an amino acid sequence at least 85% identical to SEQ ID NO: 30, and the VH domain of the present CHS7 antibody (e.g., CHS7 IgG or CHS7 scFv) comprises an amino acid sequence at least 85% identical to SEQ ID NO: 31. According to some preferred embodiments, the VL and VH domains of the present CHS7 antibody (e.g., CHS7 IgG or CHS7 scFv) respectively comprise amino acid sequences at least 90% identical to SEQ ID NOs: 30 and 31. More preferably, the VL and VH domains of the present CHS7 antibody (e.g., CHS7 IgG or CHS7 scFv) respectively comprise amino acid sequences at least 95% identical to SEQ ID NOs: 30 and 31. In one exemplary embodiment, the VL domain of the present CHS7 antibody (e.g., CHS7 IgG or CHS7 scFv) comprises the amino acid sequence of SEQ ID NO: 30, and the VH domain of the present CHS7 antibody (e.g., CHS7 IgG or CHS7 scFv) comprises the amino acid sequence of SEQ ID NO: 31.

According to certain embodiments, the VL domain of the present CHS8 antibody (e.g., CHS8 IgG or CHS8 scFv) comprises an amino acid sequence at least 85% identical to SEQ ID NO: 32, and the VH domain of the present CHS8 antibody (e.g., CHS8 IgG or CHS8 scFv) comprises an amino acid sequence at least 85% identical to SEQ ID NO: 33. According to some preferred embodiments, the VL and VH domains of the present CHS8 antibody (e.g., CHS8 IgG or CHS8 scFv) respectively comprise amino acid sequences at least 90% identical to SEQ ID NOs: 32 and 33. More preferably, the VL and VH domains of the present CHS8 antibody (e.g., CHS8 IgG or CHS8 scFv) respectively comprise amino acid sequences at least 95% identical to SEQ ID NOs: 32 and 33. In one exemplary embodiment, the VL domain of the present CHS8 antibody (e.g., CHS8 IgG or CHS8 scFv) comprises the amino acid sequence of SEQ ID NO: 32, and the VH domain of the present CHS8 antibody (e.g., CHS8 IgG or CHS8 scFv) comprises the amino acid sequence of SEQ ID NO: 33.

According to alternative embodiments, the VL domain of the present ALA12 antibody (e.g., ALA12 IgG or ALA12 scFv) comprises an amino acid sequence at least 85% identical to SEQ ID NO: 34, and the VH domain of the present ALA12 antibody (e.g., ALA12 IgG or ALA12 scFv) comprises an amino acid sequence at least 85% identical to SEQ ID NO: 35. According to some preferred embodiments, the VL and VH domains of the present ALA12 antibody (e.g., ALA12 IgG or ALA12 scFv) respectively comprise amino acid sequences at least 90% identical to SEQ ID NOs: 34 and 35. More preferably, the VL and VH domains of the present ALA12 antibody (e.g., ALA12 IgG or ALA12 scFv) respectively comprise amino acid sequences at least 95% identical to SEQ ID NOs: 34 and 35. In one exemplary embodiment, the VL domain of the present ALA12 antibody (e.g., ALA12 IgG or ALA12 scFv) comprises the amino acid sequence of SEQ ID NO: 34, and the VH domain of the present ALA12 antibody (e.g., ALA12 IgG or ALA12 scFv) comprises the amino acid sequence of SEQ ID NO: 35.

According to some embodiments of the present disclosure, each of the antibody exhibits a binding affinity and/or specificity to MSLN.

### (II-4) Immunoconjugates and Pharmaceutical Compositions Comprising the Same

The present antibody is useful in constructing an ADC for treating cancers, e.g., MSLN-positive cancers. Accordingly, another aspect of the present disclosure is directed to an immunoconjugate, which, in structure, comprises an antibody (e.g., a recombinant IgG antibody or its fragment, such as an scFv) of the present disclosure, a functional motif, and a linker for connecting the antibody and the functional motif.

According to one exemplary embodiments, the antibody is antibody CHS5 (e.g., CHS5 scFv or CHS5 IgG), and comprises amino acid sequences of SEQ ID NO: 7 (CDR-L1), SEQ ID NO: 8 (CDR-L2), SEQ ID NO: 9 (CDR-L3), SEQ ID NO: 10 (CDR-H1), SEQ ID NO: 11 (CDR-H2) and SEQ ID NO: 12 (CDR-H3). According to another exemplary embodiments, the recombinant antibody/antibody fragment is antibody CHS7 (e.g., CHS7 scFv or CHS7 IgG), and comprises amino acid sequences of SEQ ID NO: 13 (CDR-L1), SEQ ID NO: 14 (CDR-L2), SEQ ID NO: 15 (CDR-L3), SEQ ID NO: 16 (CDR-H1), SEQ ID NO: 17 (CDR-H2) and SEQ ID NO: 12 (CDR-H3). According to still another exemplary embodiments, the recombinant antibody/antibody fragment is antibody CHS8 (e.g., CHS8 scFv or CHS8 IgG), and comprises amino acid sequences of SEQ ID NO: 7 (CDR-L1), SEQ ID NO: 18 (CDR-L2), SEQ ID NO: 19 (CDR-L3), SEQ ID NO: 20 (CDR-H1), SEQ ID NO: 21 (CDR-H2) and SEQ ID NO: 22 (CDR-H3). According to further another exemplary embodiments, the recombinant antibody/antibody fragment is antibody ALA12 (e.g., ALA12 scFv or ALA12 IgG), and comprises amino acid sequences of SEQ ID NO: 23 (CDR-L1), SEQ ID NO: 24 (CDR-L2), SEQ ID NO: 25 (CDR-L3), SEQ ID NO: 26 (CDR-H1), SEQ ID NO: 27 (CDR-H2) and SEQ ID NO: 12 (CDR-H3).

The functional motif comprises a therapeutic agent, and optionally, an ER retention peptide (e.g., KDEL, SEQ ID NO: 36) connected to the therapeutic agent. Depending on desired purposes, the therapeutic agent may be an immunotoxin, an immunoliposome or a cytotoxic drug. Non-limiting examples of the immunotoxin include, diphtheria A subunit, nonbinding fragments of diphtheria toxin, exotoxin A subunit, ricin A subunit, abrin A subunit, modeccin A subunit, alpha-sarcin, Aleurites fordii proteins, dianthin proteins, Phytolacca americana proteins (PAPI, PAPII, and PAP-S), Momordica charantia inhibitor, curcin, crotin, Saponaria officinalis inhibitor, gelonin, mitogellin, restrictocin, phenomycin, and enomycin. According to some preferred embodiments, the immunotoxin is exotoxin; more preferably, the immunotoxin is or is derived from Pseudomonas Exotoxin (PE) A. In one working example of the present disclosure, the immunotoxin is a truncated form of PE A subunit toxin.

Examples of the cytotoxic drug include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel, docosahexaenoic acid bound-paclitaxel (DHA-paclitaxel, Taxoprexin), polyglutamate bound-paclitaxel (PG-paclitaxel, paclitaxel poliglumex), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib, bosutinib, cediranib, dasatinib, erlotinib, gefitinib, imatinib, lapatinib, lestaurtinib, neratinib, nilotinib, semaxanib, sunitinib, toceranib, vandetanib, vatalanib, trastuzumab, bevacizumab, rituximab, cetuximab, panitumumab, ranibizumab, nilotinib, sorafenib, everolimus, alemtuzumab, gemtuzumab ozogamicin, temsirolimus, dovitinib lactate, and tivozanib), proteasome inhibitors (e.g., bortezomib), mTOR inhibitors (e.g., rapamycin, temsirolimus, everolimus, and ridaforolimus), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. According to one specific example of the present disclosure, the cytotoxic drug is auristatin or a derivative thereof (e.g., MMAE or MMAF).

The immunoliposome comprises at least one therapeutic agent (e.g., immunotoxin and/or cytotoxic drug) encapsulated in the liposome structure. The liposome may be a large unilamellar vesicle (LUV), a multilamellar vesicle (MLV) or a small unilamellar vesicle (SUV), depending on desired purposes.

The linker may be a valine-citrulline (vc) dipeptide, a polypeptide, a DNA, a RNA, an aliphatic chain, or an adaptor. According to some embodiments of the present disclosure, the linker is a valine-citrulline dipeptide; in these embodiments, the immunoconjugate is present in the form of IgG-vc-drug, in which the therapeutic agent (e.g., MMAE) is connected to the cysteine residues of the IgG antibody via the valine-citrulline dipeptide. According to certain embodiments of the present disclosure, the linker is a polypeptide having the amino acid sequence of “ASAAGGSGT” (SEQ ID NO: 37); the thus-produced immunoconjugate comprises an antibody, a polypeptide and a functional motif PE38DKEL, in sequence, from N-terminus to C-terminus, in which the functional motif PE38DKEL comprises a truncated form of PE A subunit toxin (i.e., PE38) and a ER retention peptide (i.e., KDEL; SEQ ID NO: 36). According to alternative embodiments of the present disclosure, the linker is an adaptor comprising one or more AL module, in which each AL module comprises a protein A fragment at the N-terminus, a protein L fragment at the C-terminus, and a polypeptide connecting the protein A and protein L fragments. In one working examples, the IgG antibody is connected to the functional motif (e.g., PE38KDEL) via one AL module, which comprises the amino acid sequence of SEQ ID NO: 38.

The method for constructing an ADC is known by the person having ordinary skill in the art. Alternatively, the present immunoconjugate can be prepared in accordance with method described in U.S. Pat. No. 10,752,673 B2 or 10,562,976 B2.

Also disclosed therein is a pharmaceutical composition comprising the immunoconjugate in accordance with any embodiment of the present disclosure, and a pharmaceutically acceptable excipient.

Generally, the present immunoconjugate is present in the pharmaceutical composition at a level of about 0.01% to 99.9% by weight, based on the total weight of the pharmaceutical composition. In some embodiments, the present immunoconjugate is present at a level of at least 0.1% by weight, based on the total weight of the pharmaceutical composition. In certain embodiments, the present immunoconjugate is present at a level of at least 5% by weight, based on the total weight of the pharmaceutical composition. In still other embodiments, the present immunoconjugate is present at a level of at least 10% by weight, based on the total weight of the pharmaceutical composition. In still yet other embodiments, the present immunoconjugate is present at a level of at least 25% by weight, based on the total weight of the pharmaceutical composition.

Preferably, the present pharmaceutical composition is formulated into liquid forms, such as solutions, suppositories, and injections. As such, administration of the present immunoconjugate can be achieved in a suitable way, such as intravenous, intraarterial, intraperitoneal, or intratumoral injection. In pharmaceutical dosage forms, the present immunoconjugate may be administered alone or in combination with other known pharmaceutically active agent to treat diseases and conditions caused by/associated with cancers. One of skilled person in the art is familiar with the various dosage forms that are suitable for use in each route. It is to be noted that the most suitable route in any given case would depend on the nature or severity of the disease or condition being treated.

(II-5) Methods of treating cancers

Another aspect of the present disclosure pertains to a method of treating a cancer, especially an MSLN-positive cancer (i.e., the cancer having MSLN expressed thereon), in a subject. The method comprises administering to the subject an effective amount of the present immunoconjugate or pharmaceutical composition in accordance with any aspect and embodiment of the present disclosure.

In one embodiment, the subject is a mouse. To elicit a therapeutic effect in mice, about 0.1 to 1,000 mg of present immunoconjugate per Kg body weight per dose is administered (i.e., the present immunoconjugate is administered to the subject in the amount of about 0.1 to 1,000 mg per Kg body weight per dose; alternatively, in the case when the pharmaceutical composition is administered to the subject, it gives rise to about 0.1 to 1,000 mg of the present immunoconjugate per Kg body weight per dose); for example, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1,000 mg per Kg body weight per dose. Preferably, about 1 to 100 mg of the present immunoconjugate per Kg body weight per dose is administered. According to one working example, 10 to 20 mg of the present immunoconjugate per Kg body weight per dose is sufficient to elicit a tumor-specific cytotoxic response (e.g., inhibiting tumor growth) in the subject.

A skilled artisan could calculate the human equivalent dose (HED) of the present immunoconjugate, based on the doses determined from animal models. Accordingly, the effective amount of the present immunoconjugate suitable for use in a human subject may be in the range of 0.01 to 100 mg per Kg body weight per dose for human, for example, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg per Kg body weight per dose. Preferably, the effective HED is about 0.1 to 10 mg per Kg body weight per dose. In one preferred example, the effective HED is about 1 to 2 mg per Kg per dose.

The effective amount of the present immunoconjugate or the pharmaceutical composition may vary with many factors, such as the physical condition of the patient (e.g., the patient's body mass, age, or gender), the severity of the condition, the type of mammal or animal being treated, the duration of the treatment, and the nature of concurrent therapy (if any), and the specific route of administration and like factors within the knowledge and expertise of the health practitioner.

For repeated administrations over several days or longer, depending on the condition, the treatment is sustained until a desired suppression of symptoms occurs or until sufficient therapeutic levels are achieved to alleviate the cancer, or a symptom thereof. According to some embodiments of the present disclosure, the present immunoconjugate or pharmaceutical composition is administered to the subject at least 2 times, for example, 2, 3, 4, 5 or more times. The dosing frequency may be once every day, every 2 days, every 3 days, every 4 days, every 5 days, every 6 days, every week, every 2 weeks, every 3 weeks, every month, every 2 months, every 3 month, or longer. In one working example, the present immunoconjugate is administered every weeks for 3 consecutive weeks.

The immunoconjugate or pharmaceutical composition may be administered intraveneously, intraarterially, intraperitoneally, intralesionally or intratumorally. According to one embodiment, the immunoconjugate/pharmaceutical composition is intraveneously administered to the subject.

The cancer treatable with the present method is a gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma. According to one embodiment, the cancer is a gastric cancer. According to another embodiment, the cancer is a pancreatic cancer.

The subject is a mammal, such as a human, a mouse, a rat, a monkey, a sheep, a goat, a cat, a dog, a horse, or a chimpanzee. Preferably, the subject is a human.

As would be appreciated, the present method can be applied to the subject, alone or in combination with additional therapies that have some beneficial effects on the treatment of cancers. Depending on the intended/therapeutic purposes, the present method can be applied to the subject before, during, or after the administration of the additional therapies.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

Methodology for Optimizing CDR Sequences of Antibodies Derived from a Parent Antibody

In the present study, M9 scFv (the CDR-L1, CDR-L2, CDR-L3, CDR-H1, CDR-H2 and CDR-H3 respectively comprising the amino acid sequences of SEQ ID NOs: 39-44) was employed as a parent template, and six synthetic scFv libraries were respectively constructed, in which each of the synthetic scFv libraries contained degenerate codons (NNK) to diversify selected residue positions in only one CDR of M9 while leaving the rest of the M9 template sequence unchanged. These synthetic scFv libraries were individually expressed with the M13 phage display system, and the phage-displayed scFv libraries were respectively used as input for phage display selection against immobilized MSLN. While the binding mode of the selected scFv CDR-variants of M9 (i.e., the scFvs having CDRs diversified from the CDR sequences of M9) to MSLN remained locked by the constant CDRs from the M9 template, the selected sequences of the diversified CDR were expected to further enhance the local interactions of the corresponding CDR with MSLN. Following three rounds of phage display selection on MSLN-binding with each of the six phage-displayed scFv libraries, scFvs that folded properly (with positive binding to both Protein A and Protein L) and bound to MSLN were selected and sequenced for CDR sequence analysis. After the confirmation of the sequence analysis, the degenerate codon-diversified CDRs were respectively amplified by PCR from the corresponding output library of the phage display selections; the PCR-amplification primer pairs were designed with the M9 template with overlaps in the way such that another round of PCR-amplification of the mixture of the six PCR products with a pair of primers designed with the M9 template completed the scFv library on the basis of the M9 template and with the CDR sequences optimally selected to enhance local interactions between the CDRs and the M9 epitope on MSLN. This reassembled library was again expressed with the M13 phage display system and used as input for two rounds of phage display selection against MSLN. Single colonies of Escherichia coli (E. coli) harboring individual scFv phagemid from the output libraries were cultured for soluble scFv screening and characterizations. The scFvs CDR-variants of M9 that bound to Protein A, Protein L and MSLN were selected and sequenced.

Four scFv CDR-variants of M9 were selected in the present study, and respectively designated as “CHS5 scFv”, “CHS7 scFv”, “CHS8 scFv” and “ALA12 scFv”.

### Cell Lines

Human gastric carcinoma cell line NCI-N87 (ATCC CRL-5822), human lung carcinoma cell line NCI-H226 (ATCC CRL-5826), and human pancreas adenocarcinoma cell line Capan-2 (ATCC HTB-80) were purchased from American Type Culture Collection (ATCC). The NCI-N87 and NCI-H226 cells were grown in RPMI-1640 medium supplied with 10% fetal bovine serum and 1× antimycotic at 37° C. in a humidified incubator containing 5% CO₂. Capan-2 cells were grown in McCoy's 5A medium (ATCC-20-2007) supplied with 10% fetal bovine serum and 1× antimycotic at 37° C. in a humidified incubator containing 5% CO₂. OVCAR-8, OVCAR-5, IGR OV1, M14, UO-31, HOP-62, PC-3, HT-29, T-47D and SNB-19 cell lines were obtained from NCI-60 cell panel and were cultured in RPMI 1640 medium with 10% fetal bovine serum, 2 mM L-glutamine and 1× antimycotic.

Mean Fluorescence Intensity (MFI) of scFv Labeled with AL1-RFP on Cell Surface by Flow Cytometry

MSLN-expressing culture cells were used for scFv CDR-variant binding characterization with flow cytometric analysis. First, cells were scraped and went through strainer with 40-micron pore. About 2×10⁵ cells were incubated with 100 μL of scFv at 4° C. for 30 minutes, then washed once with 0.5% FBS 1×PBS (wash buffer), mixed with 1 μg AL1-RFP in 50 μL wash buffer at 4° C. for 20 minutes, and then washed twice with wash buffer. After centrifugation and resuspension, cells were analyzed for red fluorescent protein (RFP) signal by flow cytometry. Data analysis were performed by software. Mean fluorescence intensity (MFI) was used to indicate affinity of scFvs in binding to the MSLN-expressing cells.

Cytotoxicity Assay for scFvs

10⁴ cells/well were seeded in 96-well plates. 0.5 nM scFvs were pre-incubated with AL1-PE38KDEL at a molar ratio of 1:1 for 1 hour at room temperature so as to form non-covalently linked immunotoxins. scFv-AL1-PE38KDEL mixtures were added to cell culture without serum. After 4 hours of incubation at 37° C., the antibody toxin mixture was replaced by fresh normal medium with serum. After 4 days of culture at 37° C., the number of viable cells was quantified by measuring OD450. Percentage of cell viability was calculated.

### IgG1 Preparation

Antibody IgG1s were produced by recombinant IgG vector transfection to EXPI293F™ cells. EXPI293F™ cells were kept in EXPI293™ expression medium with vent-cap baffled flask for better activity. Transfection was performed using transfection Kit according to the manufacturer's instructions. After 3 days of incubation, the antibodies in supernatant was purified using Protein A resin affinity chromatography. The purity of the antibodies was analyzed by sodium dodecyl sulfate-polyacrylamide electrophoresis (SDS-PAGE), and the concentration was determined by spectrophotometer.

### Hydrophobic Interaction Chromatography (HIC)

Buffer A (potassium phosphate buffer, pH 7.2, 50 mM; 1.5 M ammonium sulfate) and buffer B (potassium phosphate buffer, pH 7.2, 50 mM) were prepared. Protein analytes were first precipitation-tested in solution of 20%:80% buffer B:A. These protein analyte solutions were centrifuged at 13,000 rpm for 3 minutes, and the protein analyte concentrations in the supernatants were determined by spectrophotometry. Protein analytes without substantial loss of expected concentration in the supernatant were analyzed with HIC. Before sample injection, the column was equilibrated with 5 mL of buffer A. 100 μL of protein analyte (0.5 mg/mL in buffer 20%:80% buffer B:A) were loaded onto the column with 1 mL of injection loop washing. The linear gradient from 20%:80% buffer B:A to 90%: 10% buffer B:A was completed in 13 mL of running volume. The column was then washed with 1 mL 90%: 10% buffer B:A, followed by washing with 4 ml of 20%:80% buffer B:A.

### IgG1-vcMMAE ADC Preparation and ADC Yield

The IgG1s were conjugated with vcMMAE through the cysteine residues on the tris(2-carboxyethyl)phosphine hydrochloride (TCEP)-reduced IgG1s. Briefly, antibody was partially reduced for 1 hour at room temperature with tris(2-carboxyethyl)phosphine (TECP, Sigma-Aldrich) at 2 equivalent of reductant-to-IgG1 molar ratio. N-acetylcysteine was used to quench the reaction at room temperature for 30 minutes. The quenched reaction mixture was desalted by gel filtration with a 5 mL desalting column; the buffer was changed into phosphate-buffered saline (PBS), and the ADC product was concentrated by centrifugal ultrafiltration. The ADC solutions were filtered through a 0.2 μm filter and stored at 4° C. The ADC products were analyzed by SDS-PAGE. The ADC yield of the IgG1-vcMMAE conjugation was calculated as the percentage of the IgG1 in the output ADC product over the total input IgG1.

Measurements of EC₅₀ with ELISA for MSLN Binding

For determining the EC₅₀ of purified human IgG1s and IgG1-vcMMAE binding to MSLN, MSLN (0.3 μg per well) was coated in PBS buffer (pH 7.4) on 96-well plates overnight at 4° C., and blocked with 5% milk in PBST (0.1% (v/v) TWEEN® 20) for 1 hour. In the meantime, antibodies/ADCs in PBST with 5% milk were prepared at 11 concentrations by two-fold serial dilution, and then added 100 μL diluted samples to the plate. After 1 hour of binding and washing three times with PBST, 100 μL 1:5,000 anti-human IgG horseradish peroxidase antibody was added for 1 hour incubation. After washing three times with PBST and twice with PBS buffer, the plate was developed for 3 minutes with 3,3′,5,5′-tetramethyl-benzidine peroxidase substrate (TMB substrate), quenched with 1.0 M HCl, and read spectrophotometrically at 450 nm. The EC₅₀ (nM) was calculated.

Cytotoxicity (IC₅₀) measurements for IgG1-veMMAEs an IgG1-AL1-PE38KDEL

N87 cells (1×10⁴) were seeded in 96-well plates for the IC₅₀ measurements of cell viability. For IgG1-AL1-PE38KDEL, IgGs were pre-incubated with AL1-PE38KDEL at a molar ratio of 1:2 for IgG1:AL1-PE38KDEL for 1 hour at room temperature. This procedure allows the formation of non-covalently linked immunotoxins. IgG1-AL1-PE38KDEL mixtures or purified IgG1-vcMMAE solutions were added to culture medium without serum. After incubation at 37° C. for 16 hours, the medium was replaced by fresh normal medium with serum, and the cytotoxicity was assessed using WST-1 reagent after 72 hours of incubation at 37° C. Values were normalized by corresponding PBS-treated control cells as 100% viability and IC₅₀ values were calculated by software.

### Cytotoxic Specificity Assay

Approximately 2×10+ cells of N87, IGR-OV1, M14, UO-31, HOP-62, PC-3, HT-29, T-47D and SNB-19 cell lines were seeded in 96-well plates for each well. Different concentrations of IgG1-vcMMAE ADCs were directly added to the 10% FBS culture medium. 4 days after the ADC treatment, 10 μL of WST-1 solution was added to each well. After 5 hours incubation at 37° C., absorbance at 450 nm was determined. The percentage of cell viability was quantified by the determined OD 450 nm.

### Xenograft Mouse Model Treatment

8-week-old male NOD/SCID mice were subcutaneously injected with tumor cells. Each mouse was implanted with 1×10⁶ N87 cells or 3×10⁶ Capan-2 cells, and treated with anti-MSLN IgG1-vcMMAEs post 14 days or 21 days, respectively. When the tumors reached suitable tumor size of 80-100 mm³, the mice were randomly assigned into control and treatment groups and dosing was started. Anti-MSLN IgG1-vcMMAEs (15 mg/kg) were intravenous injected into tail vein once a week for a total of three doses. Tumor volume and body weight of each xenograft mouse were continuously measured until day 35 post treatment of anti-MSLN IgG1-vcMMAEs. Endpoint tumor volume at day 35 for each of experimental subjects were plotted for each treatment group. Tumor volume was calculated using the ellipsoid formula: length×width×height×0.523.

### Example 1 CDR Sequence Preferences Responsible for the Antigen Recognition of Anti-MSLN Antibody CDR-Variants of M9

As described in Materials and Methods, anti-MSLN antibody M9 was used as an antibody template to optimize CDR sequences for anti-MSLN ADCs. The MSLN-positive scFv CDR-variants of M9 from the output libraries of the phage display selections were used to derive the CDR sequence preference profile of M9. The CDR sequences of the MSLN-positive scFv CDR-variants of M9 (i.e., the CHS5 scFv, CHS7 scFv, CHS8 scFv and ALA12 scFv), which were defined by the positive binding of the soluble scFv to MSLN, Protein L and Protein A (Protein L/A binding indicates proper folding of the scFv structure), were summarized in Table 1 above. The analytic results indicated that CDRL3 and CDRH3 were prominent in the sequence preference profile of M9, and hence some of the residues in these two CDRs (in particular, L91-Y and L94-W in CDRL3 and H97-Y and H98-W in CDRH3) formed the functional paratope on the scFv CDR-variants of M9 (data not shown). The conservativeness in the CDR sequence preference of L3 and H3 reflected the conserved local CDR-antigen interactions, suggesting that the scFv CDR-variants of M9 bound to the same epitope on MSLN as that of M9. The rest of the CDRs were relatively less conserved in sequence preferences (data not shown), suggesting that these regions were in the peripheral area of the M9-MSLN functional interface, and thus were less stringent in sequence requirements of the scFv CDR-variants for MSLN recognition.

### Example 2 scFv Candidates for ADC Development Assessed with High Throughput In Vitro Cytotoxicity and Flow Cytometry Binding Assays

The MSLN-positive scFv CDR-variants of M9 (i.e., the CHS5 scFv, CHS7 scFv, CHS8 scFv and ALA12 scFv) were further subjected to specificity and affinity assessments; the sequences of these scFv CDR-variants of M9 were summarized in Table 1 above. Each of the scFvs was secreted in the medium of each individual E. coli cell culture harboring the corresponding monoclonal scFv phagemid. To assess these scFvs as targeting modules for anti-MSLN ADCs, the soluble scFvs were respectively non-covalently conjugated to AL1-RFP (Protein A-Protein L-red fluorescence protein fusion protein) and AL1-PE38KDEL (Protein A-Protein L-Pseudomonas exotoxin A) for mean fluorescence intensity (MFI) and cytotoxicity measurements. Both measurements were carried out on cultured human cancer cell lines of N87 and H226, each of which had MSLN expressed on the cell surface. The AL1-RFP and AL1-PE38KDEL were fusion proteins with single polypeptide chain containing Protein A, Protein L and RFP or PE38KDEL respectively; Protein A and Protein L in the fusion proteins non-covalently bound to the heavy chain and light chain of the scFv respectively with 1:1 molar ratio in nM affinity. The binding of Protein A and Protein L to the natively folded scFv structure did not interfere with the paratope-epitope interface of the scFv-antigen interaction.

The in vitro assessments of the selected scFv CDR-variants of M9 indicated that specific scFv-MSLN binding resulted in receptor mediated endocytosis of the scFv. AL1-RFP MFI measurements and AL1-PE38KDEL cytotoxicity measurements with N87 cells were positively correlated with the corresponding measurements using H226 cells with R²=0.87 and 0.74, respectively (data not shown). The high correlations indicated that these MSLN-positive scFvs bound to the MSLN expressed on the cell surface of both cell lines, and that the cytotoxicities of the scFv-AL1-PE38KDEL immunotoxins were attributed to the binding of the scFv to the cell surface MSLN, rather than non-specific cytotoxic effect independent to the scFv-MSLN binding. These two implications were further illustrated with the plots of the MFI of scFv-AL1-RFP versus the cytotoxicity of scFv-AL1-PE38KDEL (data not shown). The negative correlations between the MSLN binding and the cell viability for the scFv CDR-variants of M9 with R²=0.54 and 0.68 respectively for N87 and H226 cells (data not shown) were consistent with the most likely implication that the scFv-AL1-PE38KDEL cytotoxicity was due to cell surface receptor mediated endocytosis of the immunotoxin through the specific scFv-MSLN binding. The cytotoxicity of the immunotoxins on H226 cells was more potent in comparison with that on N87 cells (data not shown), most likely due to the fact that H226 cells expressed more MSLN on the cell surface than N87 cells did, as judged by the higher absolute MFIs measured with the scFv-AL1-RFPs on H226 cells (data not shown).

### Example 3 Antibody Solubility in Aqueous Environment as a Critical Determinant for the Antibodies as Targeting Modules for Anti-MSLN ADCs

Based on the scFv-MSLN interaction data, 4 scFv CDR-variants of M9 (i.e., the CHS5 scFv, CHS7 scFv, CHS8 scFv and ALA12 scFv) were reformatted with human IgG1 framework for further evaluation of specificity and efficacy of these IgG1s as targeting modules for anti-MSLN ADCs. These scFvs were selected mostly with both strong cell surface MSLN binding and potent immunotoxin cytotoxicity. The thus-produced IgGs were respectively designated as “CHS5 IgG”, “CHS7 IgG”, “CHS8 IgG” and “ALA12 IgG”.

Since the solubilities of the IgG1s as ADC candidates were expected to be critical for the ADCs' preparation and efficacy, a relative hydrophilicity score (RH-score) was calculated by equations (1) and (2) for a query scFv CDR-variant of M9 to anticipate the solubility of the IgG1 reformatted from the query scFv in comparison with that of IgG1-M9:

H-score (query scFv)=1×(number of type I amino acid sequence in the CDRs of the query scFv) +2(number of type II amino acid sequence in the CDRs of the query scFv) (1),

RH-score (query scFv)=H-score (query scFv)−H-score (M9 scFv) (2);

wherein type I and type II amino acid sequences consisted of the amino acid sequence of SEQ ID NOs: 45 and 46, respectively.

The RH-scores of the MSLN-positive scFv CDR-variants of M9 were plotted against the CamSol scores calculated with the corresponding scFv sequences; the CamSol scores were predicted with the CamSol computer algorithm, which has been validated, with accuracy to an extent, in predicting actual protein solubility in aqueous solution based on the protein sequence. According to the distribution curves, more than 90% of the MSLN-positive scFv CDR-variants of M9 had higher RH-score and higher predicted solubility in comparison with those of the parent M9 scFv (data not shown), indicating that the optimized CDR sequences for MSLN binding were more hydrophilic and predicted to be more soluble in water. The positive correlation (R²=0.58 and P-value=1.5×10⁻⁷¹) between the RH-score and the CamSol score indicated that the predicted solubility of the antibodies by CamSol was semi-quantitatively related to the number of hydrophilic/charged amino acid types in the CDRs of the scFv CDR-variants of M9, suggesting that, as expected, increasing the number of the hydrophilic/charged residues in the CDRs of an antibody was expected to increase the solubility of the antibody in aqueous environment.

The solubilities of the IgG1s reformatted from the selected scFv CDR-variants of M9 were measured by the retention time of HIC (hydrophobic interaction chromatography), which is a hydrophobicity indicator for the analyte protein, and is expected to be negatively correlated with the protein solubility in water. The HIC retention times were plotted against the RH-scores of the reformatted IgG1s. The HIC retention time was negatively correlated with the RH-score with R²=0.40 (P-value=1.7×10⁻⁵) (data not shown), in agreement with the expectation that the IgG1s with increasingly large RH-score were increasingly more hydrophilic and hence were predicted to be more soluble in aqueous solution in comparison with IgG1-M9 (data not shown).

IgG1s were conjugated with vcMMAE (monomethyl auristatin E linked to the IgG1 via valine-citrulline dipeptide cathepsin-cleavable linker) through the cysteines of the reduced disulfide bonds on the IgG1s. All the ADCs, for which the DARs (drug-antibody ratios) were measured with hydrophobic interaction chromatography (HIC) (Table 2), did not aggregate. The HIC analyses of the IgG1-vcMMAEs on a butyl-NPR column yielded peaks corresponding to different vcMMAE:IgG1 ratios, and the distributions of the peaks were used to calculate the DARs for the IgG1-vcMMAEs (Table 2).

TABLE 2
Binding and cytotoxicity characterizations of specified IgGs

| | M9 | CHS5 | CHS7 | CHS8 | ALA12 |
|---|---|---|---|---|---|
| Viability (%) - H226 | 56.7 | 21.02 | 8.67 | 72.53 | 9.1 |
| Viability (%) - N87 | 80.6 | 46.08 | 48.33 | 76.1 | 44.8 |
| CamSol score | −0.16 | 0.92 | 1.08 | 1.18 | 0.07 |
| RH-score | 0 | 13 | 11 | 14 | 5 |
| MFI (%) - H226 | 69 | 70.53 | 90.11 | 30.99 | 89 |
| MFI (%) - N87 | 64 | 77.84 | 87.03 | 31.89 | 100 |
| ADC yield (%) | 17.5 | 1.1 | 0.93 | 1.02 | 93.9 |
| HIC retention time | 18.0 | 3.8 | 7.7 | 4.2 | 5.7 |
| (minutes) | | | | | |
| DAR | ND | 2.56 | 3.21 | 2.63 | 2.72 |
| IgG1 EC₅₀ (ng/ml) | 3.15 | 4.67 | 10.3 | 5.05 | 8.37 |
| IgG1-vcMMAE EC₅₀ | 4.86 | 9.56 | 16.27 | 9.26 | 10.49 |
| (ng/mL) | | | | | |
| IgG1-AL1-PE38KDEL | 0.39 | 0.06 | 0.06 | 0.01 | 0.1 |
| IC₅₀ (nM) | | | | | |
| IgG1-vcMMAE | 78.13 | 15.03 | 25.95 | 24.57 | 39.94 |
| IC₅₀ (nM) | | | | | |

ND: Non-detected.

Antibody hydrophilicity promoted ADC yield and DAR for vcMMAE conjugation to IgG1s. The ADC yield increased with increasing RH-score, but the positive correlation was weak with R²=0.10 (P-value=0.018) (data not shown). The DAR (drug-antibody ratio) of the ADCs also increased with increasing RH-score with R²=0.15 (P-value=0.10) (data not shown). Both results suggested that the hydrophilicity of the IgG1s facilitated the vcMMAE-to-IgG1 conjugation, albeit with weak positive correlation. Moreover, the half maximal effective concentrations for MSLN binding (MSLN-EC₅₀'s) of the IgG1-vcMMAEs versus the MSLN-EC₅₀'s of the IgG1s were plotted with the linear correlation of slope=1.1 and R²=0.62 (P-value=1.0×10⁻⁷). The correlation indicated that the conjugation of vcMMAE to the cysteines of the reduced disulfide bonds on the IgG1 had little impact on the binding of the IgG1 to MSLN.

Together, IgG1-M9 was not feasible as an ADC candidate because of the low ADC yield and DAR due to the overly hydrophobic CDRs. By contrast, the IgG1s reformatted from the scFvs selected with the binding and cytotoxicity characterizations (including the CHS5 IgG, CHS7 IgG, CHS8 IgG and ALA12 IgG) were likely to be feasible ADC candidates. These IgG1s conjugated with the hydrophobic drug vcMMAE exhibited higher ADC yield and DAR in comparison with those of M9 (Table 2). The feasibility of these IgG1s as candidates for ADC development was attributed to the hydrophilic/charged amino acids encoded in the CDR-variant sequences, as indicated by the relatively high RH-score in comparison with that of M9 (Table 2).

### Example 4 Potencies of the IgG1s Reformatted from the Selected scFvs as Targeting Modules for PE38-Based Immunotoxins and ADCs Conjugated with vcMMAE

The PE38KDEL-based immunotoxins and the vcMMAE-conjugated ADCs based on the IgG1s reformatted from the selected scFv CDR-variants of M9 had potent cytotoxicity in vitro against N87 cultured cells (data not shown). As expected, the half maximal inhibitory concentrations (IC₅₀'s) of the IgG1-AL1-PE38KDELs were clustered to the optimal value (about 0.1-0.2 nM) because these IgG1s were reformatted from the scFv CDR-variants of M9 selected with potent scFv-AL1-PE38KDEL cytotoxicity (data not shown). Similarly, the IC₅₀'s of IgG1-vcMMAEs were clustered between 20-80 nM (data not shown), indicating that the same set of IgG1s were also effective as the targeting modules for the vcMMAE-based ADCs against N87 cultured cells in vitro. Nevertheless, the correlation between the two sets of IC₅₀'s was insignificant (R²=0.056 and P-value=0.13), indicating that the cytotoxic mechanisms for the IgG1-AL1-PE38KDEL and IgG1-vcMMAE were not quantitatively related, and hence the potency of the vcMMAE-based ADCs could only be qualitatively inferred from the cytotoxicity of the PE38KDEL-based immunotoxins. Although the IC₅₀'s of the IgG1-vcMMAEs were expected to be related to the corresponding DARs of the ADCs, the correlation of DAR versus IC₅₀ was insignificant (data not shown), indicating that the IC₅₀'s of the ADCs were dependent on other factors, such as IgG1-MSLN interaction affinity, in addition to the DARs.

Although the IC₅₀ measurements indicated that the IgG1-AL1-PE38KDEL immunotoxins were about 1-2 orders of magnitude more potent than the IgG1-vcMMAEs (data not shown), the systemic toxicities of these immunotoxins in animal disease models had discouraged further development of these immunotoxins as therapeutics against tumors. Thus, four IgG1-vcMMAEs, i.e., CHS5-vcMMAE, CHS7-vcMMAE, CHS8-vcMMAE and ALA12-vcMMAE, were subjected to in vivo validation as cancer therapeutics in the following sections. These IgG1s were selected because of their high ADC potency, high ADC yield and DAR (data not shown), likely due to the high hydrophilicity of the CDRs in the IgG1s, as reflected by the high RH-scores and short HIC retention times for these IgG1s (Table 2). The high affinity and specificity of these IgG1 were attributed to the highly conserved aromatic residues in the CDRs of the variable domains (data not shown).

### Example 5 Specificity of the M9-Derived MSLN-Positive IgG1s in Delivering Cytotoxic Payload Through Binding to the Cell Surface MSLN

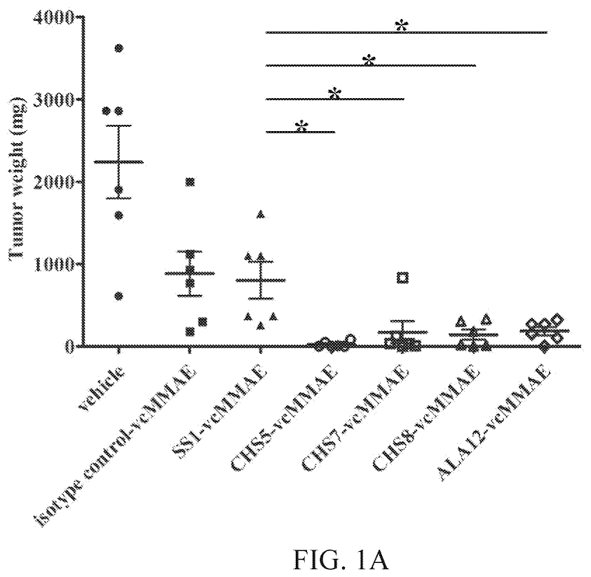

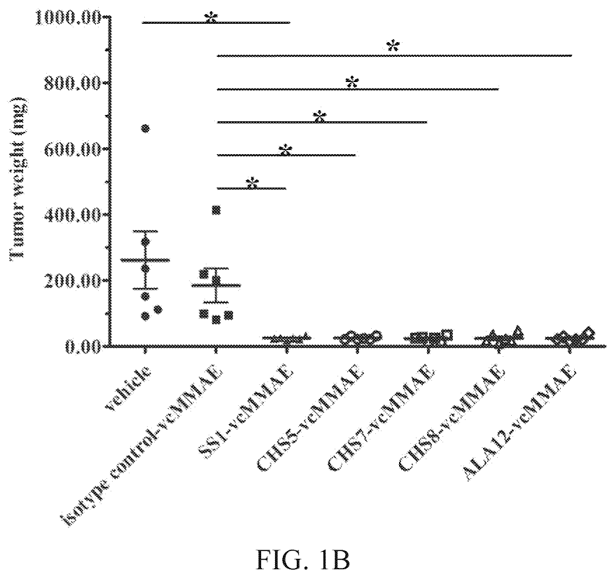

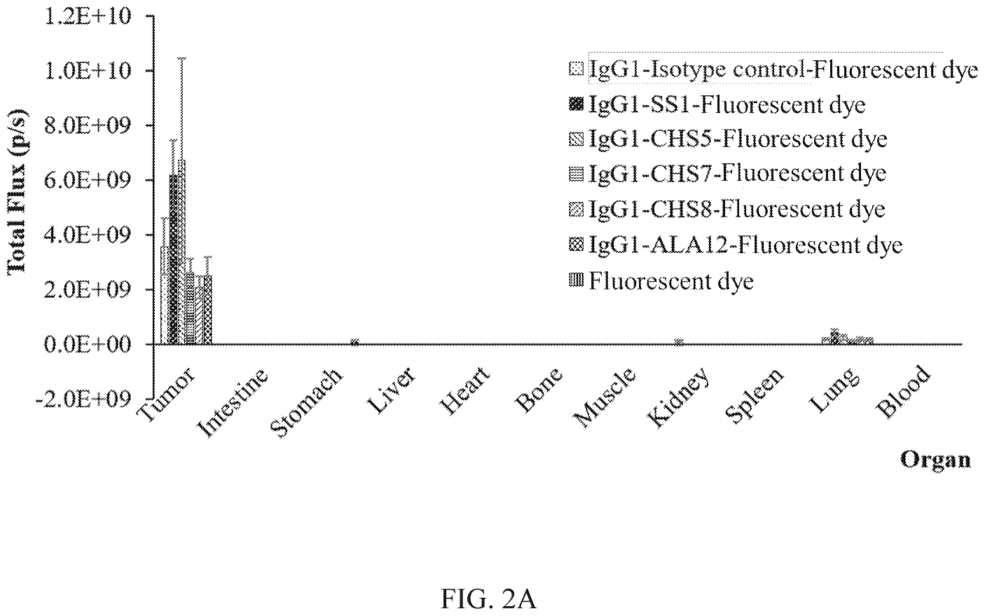

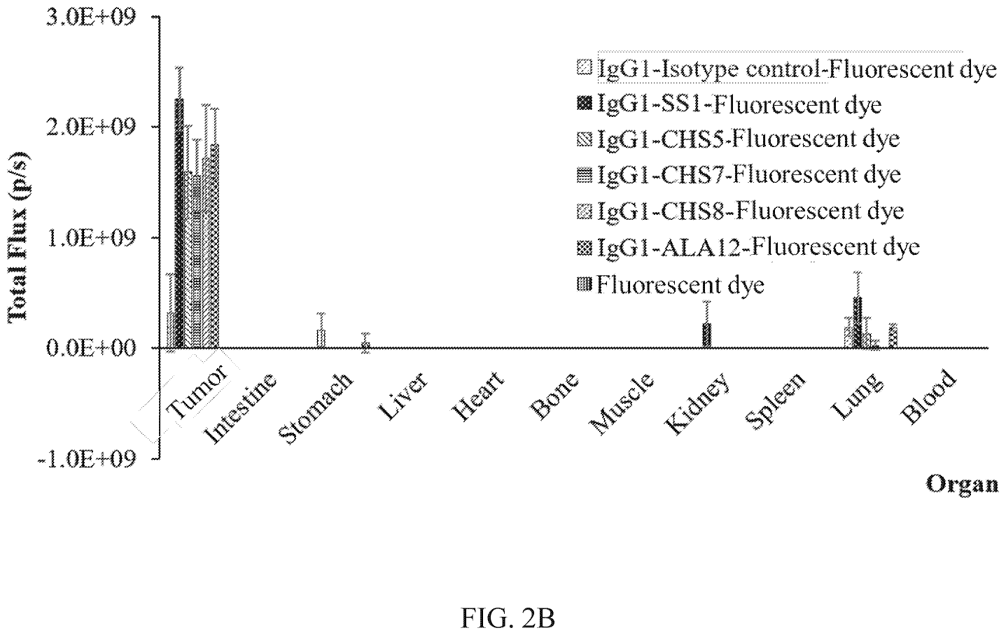

The cytotoxic specificities of the 4 selected IgG1-vcMMAEs (i.e., CHS5-vcMMAE, CHS7-vcMMAE, CHS8-vcMMAE and ALA12-vcMMAE) were analyzed and compared to that of the positive control SS1-vcMMAE, for which the anti-MSLN antibody is known to exhibit binding affinity and specificity to MSLN. Although there are many anti-MSLN antibodies known in public domain, the reason for the choice of using SS1 anti-MSLN antibody as positive control antibody was that SS1 has been documented in many publications. Moreover, SS1-based therapeutics have been registered in human trials with public information available for reproducing the antibody SS1 in the same IgG1 framework for side-by-side comparisons with the ADCs of this work in terms vcMMAE conjugation (data not shown), cell-based cytotoxicity measurements (data not shown), in vivo efficacity (FIGS. 1A and 1B) and biodistribution of the vcMMAE-based ADCs (FIGS. 2A and 2B).

A panel of 8 NCI-60 cell lines of different organ origin without MSLN expression was selected and verified the absence of MSLN expression in these culture cells in comparison with 4 MSLN-positive control cells (data not shown). The cytotoxicity of the 5 IgG1-vcMMAEs against the cell lines with/without MSLN expression was then measured in the presence of the ADC concentration of 1-, 2-, and 8-folds of the average IC₅₀ (data not shown). Other than the positive ADC cytotoxicity on the positive control N87 cells, the results indicated that significant cytotoxicity was not found for the MSLN-negative cells of different organ origin at ADC concentrations below 2-folds of the average IC₅₀. Although, two NCI-60 cell lines (M14 and, to a lesser extent, IGR-OV1) were subjected to ADC cytotoxicity at the highest ADC concentration (data not shown), the 5 IgG1-vcMMAEs had similar specificity patterns in terms of cytotoxicity against the representative panel of cell lines, suggesting a possibility that the M14 cells could express minor amount of MSLN, of which the expression level was below the detection limit of the Western Blot (data not shown). The results implied that the off-target toxicity in in vivo treatments of MSLN-positive tumors with these IgG1-vcMMAEs would be unlikely, because the efficacies of the ADCs tested were clearly associated only with the specific targeting of the cell surface MSLN by the antibodies in the ADCs.

### Example 6 In Vivo Treatments of Xenograft Tumors in Mouse Disease Models with the Anti-MSLN IgG1-vcMMAEs

Xenograft N87 (human gastric) and Capan-2 (human pancreatic) tumors in mice were treated with the four IgG1-vcMMAEs (i.e., CHS5-vcMMAE, CHS7-vcMMAE, CHS8-vcMMAE and ALA12-vcMMAE) in the experimental ADC group. In the experiment, SS1-vcMMAE served as the positive control, and isotype ADC and vehicle treatments served as the negative controls. N87 and Capan-2 cancer models were selected because both were important human cancers with limited treatment options and both cancer cells express MSLN on the cell surface (data not shown).

The data of FIGS. 1A and 1B respectively depicted the in vivo treatment results on N87 and Capan-2 xenograft tumors in mice. Both experiments indicated that the four anti-MSLN IgG1-vcMMAEs in the experimental ADC group were comparable or better in treating the N87 and Capan-2 mouse xenograft disease models in comparison with the positive control ADC (SS1-vcMMAE), and the endpoint results of almost complete eradication of xenograft tumors in several of the treatments clearly demonstrated the superior efficacies of the IgG1-vcMMAEs in the experimental ADC group as compared to those of the negative control treatments (FIGS. 1A and 1B).

Bio-distributions of the IgG1-vcMMAEs in xenograft models indicated that the control and experimental ADCs tested in the in vivo treatments above were overwhelmingly concentrated in the xenograft tumors. The 4 experimental IgG1s, along with the positive and isotype control IgG1s, were conjugated with fluorescence dye and the in vivo fluorescence imaging with these IgG1-dye conjugates indicated that the IgG1s were locally concentrated in the N87 and Capan-2 xenograft tumors one day after the administration of the IgG1-dye conjugates to the xenograft tumor mice (data not shown). Quantitative ex vivo measurements of the bio-distributions indicated that all the IgG1s targeted the N87 and Capan-2 tumors with high local concentration and low off-target propensity to all the organs (FIGS. 2A and 2B), although insignificantly minor distributions of the IgG1s in the lung of the mouse disease models were also found. The tumor-heavy bio-distributions of the IgG1s (FIGS. 2A and 2B) were consistent to these IgG1s' MSLN-specific targeting capabilities. The serum biochemical parameters in N87 and Capan-2 xenograft mice treated with these IgG1-vcMMAEs further indicated that the in vivo treatments with these IgG1-vcMMAEs were accompanied with undetectable off-target toxicities (Tables 3 and 4).

TABLE 3
Effects of anti-MSLN IgG1-vc-MMAEs on serum biochemical parameters in N87 NOD/SCID mice

| N87 | ALT(U/L) | ALP(U/L) | BUN(mg/dL) | CRE(mg/dL) | ALT/ALP |
|---|---|---|---|---|---|
| PBS | 38.17 ± 9.21 | 143.67 ± 37.73 | 26.18 ± 1.11 | 0.58 ± 0.16 | 0.27 |
| Isotype control-vcMMAE | 32.67 ± 19.05 | 99.00 ± 22.18 | 21.15 ± 2.31 | 0.77 ± 0.06 | 0.33 |
| SS1-vcMMAE | 38.50 ± 45.18 | 109.17 ± 23.08 | 21.02 ± 1.45 | 0.57 ± 0.26 | 0.35 |
| CHS5-vcMMAE | 20.67 ± 3.09 | 104.67 ± 14.21 | 20.88 ± 1.83 | 0.57 ± 0.16 | 0.20 |
| CHS7-vcMMAE | 20.67 ± 2.56 | 99.83 ± 8.55 | 21.32 ± 1.68 | 0.79 ± 0.07 | 0.21 |
| CHS8-vcMMAE | 22.83 ± 5.84 | 105.33 ± 13.61 | 24.55 ± 1.66 | 0.48 ± 0.11 | 0.22 |
| ALA12-vcMMAE | 21.17 ± 4.37 | 94.50 ± 8.52 | 21.57 ± 1.30 | 0.58 ± 0.16 | 0.22 |

End point represents mean ± SD for 6 mice.
ALT (alanine aminotransferase), ALP (alkaline phosphatase), BUN (blood urea nitrogen), CRE (creatinine).

TABLE 4
Effects of anti-MSLN IgG1-vcMMAEs on serum biochemical
parameters in Capan-2 NOD/SCID mice

| Capan2 | ALT(U/L) | ALP(U/L) | BUN(mg/dL) | CRE(mg/dL) | ALT/ALP |
|---|---|---|---|---|---|
| PBS | 16.33 ± 1.80 | 75.83 ± 17.32 | 25.13 ± 4.32 | 0.79 ± 0.12 | 0.22 |
| Isotype control-vcMMAE | 19.33 ± 8.42 | 88.17 ± 9.99 | 20.75 ± 1.30 | 0.65 ± 0.23 | 0.22 |
| SS1-vcMMAE | 26.67 ± 13.91 | 102.50 ± 25.17 | 22.53 ± 11.65 | 0.82 ± 0.29 | 0.26 |
| CHS5-vcMMAE | 18.83 ± 1.67 | 98.67 ± 7.87 | 21.88 ± 0.75 | 0.77 ± 0.09 | 0.19 |
| CHS7-vcMMAE | 16.67 ± 2.36 | 107.17 ± 23.13 | 21.82 ± 2.49 | 0.64 ± 0.06 | 0.16 |
| CHS8-vcMMAE | 16.00 ± 1.00 | 87.00 ± 9.18 | 22.30 ± 1.33 | 0.64 ± 0.14 | 0.18 |
| ALA12-vcMMAE | 18.50 ± 2.29 | 92.00 ± 19.51 | 21.88 ± 2.05 | 0.62 ± 0.14 | 0.18 |

End point represents mean ± SD for 6 mice.
ALT (alanine aminotransferase), ALP (alkaline phosphatase), BUN (blood urea nitrogen), CRE (creatinine).

Less effective treatments with partial tumor eradication in N87 disease models by the positive control and some of the IgG1-vcMMAEs in the experimental ADC group could result from the extreme tumor burden of the N87 xenograft mice during the ADC treatments. In comparison with the Capan-2 tumors with relatively less aggressive growth rate (FIGS. 1B and 2B), the highly aggressive growth of the N87 xenograft tumors in mice could also account for the intake of the isotype control IgG1 in the N87 tumor (FIG. 2A), explaining the partial effectiveness for the isotype control ADC in treating the N87 tumor disease models (FIG. 1A).

Overall, the 4 IgG1-vcMMAEs in the experimental ADC group were effective ADC therapeutics with non-detectable off-target toxicity in treating N87 and Capan-2 xenograft tumors in mice. In addition, the in vivo treatments with CHS5-vcMMAE were of particular interest because of its superior efficacies in tumor eradication in both tumor disease models. Although IgG1-CHS5's affinity to MSLN was not the highest among the IgG1s in the experimental ADC group and the DAR of CHS5-vcMMAE (2.56) was the lowest among the ADCs used in the in vivo treatments, its hydrophilicity, ADC yield, DAR, in vitro cytotoxicity of CHS5-vcMMAE, and cell surface MSLN-targeting specificity were all the most superior among the IgG1s in the experimental ADC group. These results highlight the importance of the collection of the characterizations in determining the efficacy and specificity of the candidate ADCs in the in vivo treatments leading to tumor eradication.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A phage-displayed single-chain variable fragment (scFv) library comprising a plurality of phage-displayed scFvs, wherein each of the plurality of phage-displayed scFvs comprises a first light chain complementarity determining region (CDR-L1), a second light chain CDR (CDR-L2), a third light chain CDR (CDR-L3), a first heavy chain CDR (CDR-H1), a second heavy chain CDR (CDR-H2) and a third heavy chain CDR (CDR-H3),
wherein,
the CDR-L1 is encoded by a first coding sequence comprising the nucleic acid sequence of SEQ ID NO: 1, the CDR-L2 is encoded by a second coding sequence comprising the nucleic acid sequence of SEQ ID NO: 2, the CDR-L3 is encoded by a third coding sequence comprising the nucleic acid sequence of SEQ ID NO: 3, the CDR-H1 is encoded by a fourth coding sequence comprising the nucleic acid sequence of SEQ ID NO: 4, the CDR-H2 is encoded by a fifth coding sequence comprising the nucleic acid sequence of SEQ ID NO: 5, and the CDR-H3 is encoded by a sixth coding sequence comprising the nucleic acid sequence of SEQ ID NO: 6.

2. The phage-displayed scFv library of claim 1, wherein the phage is a M13 phage or a T7 phage.

3. The phage-displayed scFv library of claim 1, wherein the phage-displayed scFv library comprises a first, a second, a third and a fourth phage-displayed scFvs, wherein
the CDR-L1, CDR-L2, CDR-L3, CDR-H1, CDR-H2 and CDR-H3 of the first phage-displayed scFv respectively comprise the amino acid sequences of SEQ ID NOs: 7, 8, 9, 10, 11 and 12;
the CDR-L1, CDR-L2, CDR-L3, CDR-H1, CDR-H2 and CDR-H3 of the second phage-displayed scFv respectively comprise the amino acid sequences of SEQ ID NOs: 13, 14, 15, 16, 17 and 12;
the CDR-L1, CDR-L2, CDR-L3, CDR-H1, CDR-H2 and CDR-H3 of the third phage-displayed scFv respectively comprise the amino acid sequences of SEQ ID NOs: 7, 18, 19, 20, 21 and 22; and
the CDR-L1, CDR-L2, CDR-L3, CDR-H1, CDR-H2 and CDR-H3 of the fourth phage-displayed scFv respectively comprise the amino acid sequences of SEQ ID NOs: 23, 24, 25, 26, 27 and 12.

4. A recombinant antibody, comprising a light chain variable (VL) domain and a heavy chain variable (VH) domain, wherein
the VL domain comprises the amino acid sequences of SEQ ID NOs: 7-9, and the VH domain comprises the amino acid sequences of SEQ ID NOs: 10-12;
the VL domain comprises the amino acid sequences of SEQ ID NOs: 13-15, and the VH domain comprises the amino acid sequences of SEQ ID NOs: 16, 17 and 12;
the VL domain comprises the amino acid sequences of SEQ ID NOs: 7, 18 and 19, and the VH domain comprises the amino acid sequences of SEQ ID NOs: 20-22; or
the VL domain comprises the amino acid sequences of SEQ ID NOs: 23-25, and the VH domain comprises the amino acid sequences of SEQ ID NOs: 26, 27 and 12.

5. The recombinant antibody of claim 4, wherein
the VL domain comprises an amino acid sequence at least 85% identical to SEQ ID NO: 28, and the VH domain comprises an amino acid sequence at least 85% identical to SEQ ID NO: 29;
the VL domain comprises an amino acid sequence at least 85% identical to SEQ ID NO: 30, and the VH domain comprises an amino acid sequence at least 85% identical to SEQ ID NO: 31;
the VL domain comprises an amino acid sequence at least 85% identical to SEQ ID NO: 32, and the VH domain comprises an amino acid sequence at least 85% identical to SEQ ID NO: 33; or
the VL domain comprises an amino acid sequence at least 85% identical to SEQ ID NO: 34, and the VH domain comprises an amino acid sequence at least 85% identical to SEQ ID NO: 35.

6. The recombinant antibody of claim 5, wherein
the VL domain comprises an amino acid sequence 100% identical to SEQ ID NO: 28, and the VH domain comprises an amino acid sequence 100% identical to SEQ ID NO: 29;
the VL domain comprises an amino acid sequence 100% identical to SEQ ID NO: 30, and the VH domain comprises an amino acid sequence 100% identical to SEQ ID NO: 31;
the VL domain comprises an amino acid sequence 100% identical to SEQ ID NO: 32, and the VH domain comprises an amino acid sequence 100% identical to SEQ ID NO: 33; or
the VL domain comprises an amino acid sequence 100% identical to SEQ ID NO: 34, and the VH domain comprises an amino acid sequence 100% identical to SEQ ID NO: 35.

7. An immunoconjugate comprising the recombinant antibody of claim 4, a functional motif, and a linker connecting the recombinant antibody to the functional motif.

8. The immunoconjugate of claim 7, wherein the functional motif comprises an immunotoxin or a cytotoxic drug.

9. The immunoconjugate of claim 8, wherein the immunotoxin is an exotoxin.

10. The immunoconjugate of claim 9, wherein the exotoxin is or is derived from Pseudomonas Exotoxin (PE) A.

11. The immunoconjugate of claim 9, wherein the functional motif further comprises an endoplasmic reticulum (ER) retention peptide connected with the exotoxin.

12. The immunoconjugate of claim 11, wherein the ER retention peptide comprises the amino acid sequence of SEQ ID NO: 36.

13. The immunoconjugate of claim 8, wherein the cytotoxic drug is auristatin or a derivative thereof.

14. The immunoconjugate of claim 13, wherein the cytotoxic drug is monomethyl auristatin E.

15. The immunoconjugate of claim 7, wherein the linker is,
a valine-citrulline dipeptide;
a first polypeptide comprising the amino acid sequence of SEQ ID NO: 37; or
an adaptor comprising at least one AL module, wherein each AL module comprises a protein A fragment at the N-terminus, a protein L fragment at the C-terminus, and a second polypeptide connecting the protein A and protein L fragments.

16. The immunoconjugate of claim 15, wherein the adaptor comprises the amino acid sequence of SEQ ID NO: 38.

17. (canceled)

18. A method of treating a cancer in a subject, comprising administering to the subject an effective amount of the immunoconjugate of claim 7.

19. The method of claim 18, wherein the cancer has MSLN expressed thereon.

20. The method of claim 19, wherein the cancer is gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma.

21. The method of claim 20, wherein the cancer is the gastric or the pancreatic cancer.

22. (canceled)
